(19) United States
(12) Patent Application Publication
(10) Pub. No.: US 2026/0334437 A1
(43) Pub. Date: Oct. 29, 2026

(54) PET SOUNDPROOF CRATE FOR OFFICE ENVIRONMENTS AND PET MANAGEMENT SYSTEM

(71) Applicant: Shaoxing Unibooth Intelligent Technology Co., Ltd, Shaoxing (CN)

(72) Inventors: Zhongxiu ZHU, Shaoxing (CN); Wentong HUANG, Shaoxing (CN); Shenggui XIE, Shaoxing (CN)

(21) Appl. No.: 19/263,539

(22) Filed: Jul. 9, 2025

(30) Foreign Application Priority Data
Apr. 24, 2025 (CN) 202510521695.6

(51) Int. Cl. A01K 29/00; A01K 1/03; A01K 13/00

(52) U.S. Cl. CPC A01K 29/005; A01K 1/033; A01K 13/00

(57) ABSTRACT

A pet soundproof crate for office environments and a pet management system are provided. The pet soundproof crate for office environments includes a plurality of outer walls enclosing a receiving cavity, and at least some of the outer walls defined as soundproof walls; a display screen; a pet living condition detection device for detecting pet's living conditions, and the display screen being configured to display the pet's living conditions; and an environmental adjustment device for adjusting the internal environment of the pet soundproof crate for office environments according to the pet’s living conditions. In the embodiment of the present application, sounds made by the pet is prevented from disrupting office order and office noise is avoided to affect mood of the pet. A comfortable environment for the pet can be maintained, thereby enhancing the pet's well-being and enabling intelligent pet management.

### CROSS-REFERENCE TO RELATED APPLICATIONS

The present application claims priority of Chinese Patent Application No. 202510521695.6, filed on Apr. 24, 2025, the entire contents of which are hereby incorporated by reference.

### TECHNICAL FIELD

The present application relates to the technical field of pet management equipment, and in particular to a pet soundproof crate for office environments and a pet management system.

### BACKGROUND

Pets, as companions to humans, contribute to happier and healthier lives. Interaction with pets enriches human life, reduces stress, improves work efficiency, and helps cultivate children's sense of responsibility and social skills. Thus, keeping pets can make our lives healthier and more fulfilling.

With the increasing popularity of pet-friendly office environments, employees wish to bring their pets to the workplace. However, pet barking, odors, and behavioral activities may disrupt office order and negatively impact non-pet-owning employees. Recently, designated isolation areas or ordinary pet cabins are employed, and it fails to effectively reduce noise, improve air quality, or provide intelligent management functions. Moreover, pet owners need to frequently check on their pets and adjust environment of the pets, requiring constant movement between the pet area and the workspace, which hinders their ability to focus on work. Therefore, there is an urgent need for a pet soundproof crate that has excellent performance in noise isolation, comfort, and intelligent management.

### SUMMARY

An object of the present invention is to provide a pet soundproof crate for office environments and a pet management system, which are capable of effectively reducing noise, improving the convenience of coexisting with pets in the workplace, improving the work experience, and strengthening corporate pet-friendly environment.

To achieve the above object, a pet soundproof crate for office environments is provided and includes:
a plurality of outer walls enclosing a receiving cavity, and at least some of the outer walls defined as soundproof walls;
a display screen equipped on at least one of the plurality of outer walls;
a pet living condition detection device, for detecting pet's living conditions, provided in the pet soundproof crate for office environments, and the display screen being configured to display the pet's living conditions; and
an environmental adjustment device, for adjusting the internal environment of the pet soundproof crate for office environments according to the pet’s living conditions, provided in the pet soundproof crate for office environments.

In one of the embodiments, each of the soundproof walls is a multilayer structure, and each of the soundproof walls includes a sound-absorbing cotton layer.

In one of the embodiments, the top wall is defined as the soundproof wall, the top wall further includes a soundproof panel layer, the soundproof plate layer is located at an outside of the sound-absorbing cotton layer; the bottom wall only includes the soundproof panel layer.

In one of the embodiments, the environmental adjustment device includes a fan disposed in the top wall, a cover is placed over the fan, a plurality of ventilation holes are defined in the cover to allow air to pass through, and both the sound-absorbing cotton layer and the soundproof board layer of the top wall are made of breathable materials.

In one of the embodiments, the outer walls further include a front wall and a rear wall, and at least one of the front wall and the rear wall is made of transparent material.

In one of the embodiments, the pet soundproof crate for office environments includes a frame body and the frame body is a frame structure to serve as a skeletal framework, each of the plurality of outer walls are all mounted within a corresponding mounting hole defined in the frame body; the frame body includes a main frame and a door frame movably connected to the main frame, and one of the plurality of outer walls is assembled on the door frame to form a door.

In one of the embodiments, the pet soundproof crate for office environments further includes a control module configured to obtain the pet's living conditions detected by the pet living condition detection device and to control the environmental adjustment device to adjust the environment inside the pet soundproof crate for office environments according to the pet's living conditions.

In one of the embodiments, the pet living condition detection device includes a temperature sensor configured to detect the temperature inside the crate, the environmental adjustment device includes a fan and a heating device, the temperature sensor and the heating device are connected to the control module, and the control module is configured to adjust the temperature inside the crate can be adjusted by the fan or the heating device when the temperature inside the crate is too high or too low.

In one of the embodiments, the pet living condition detection device further includes a camera for obtaining a pet's status inside the crate, and the control module is signal-connected to a mobile terminal and configured to send the pet's living conditions and/or the pet’s status to the mobile terminal.

In one of the embodiments, the pet living condition detection device further includes a sound sensor configured to detect whether the pet is making abnormal noises, the sound sensor is connected to the control module and configured to send a detected sound signals to the control module, and the control module is configured to generate an alert signal and trigger an alert when the pet makes abnormal noises.

In one of the embodiments, the control module stores information correlating pet’s sounds with pet’s emotions, and the control module is further configured to compare an obtained pet’s sound with the pre-stored information having pet’s sounds correlating with pet’s emotions to determine the pet's emotions.

In one of the embodiments, the alert signal generated by the control module is sent to the display screen to be displayed on the display screen; or the alert signal generated by the control module is sent to an external alarm device for alerting; or the alert signal generated by the module is sent to a mobile terminal for a reminder.

In one of the embodiments, the environmental adjustment device further includes a voice interaction system connected to the control module; wherein the control module is configured to receive voice messages transmitted from a mobile terminal and play them through the voice interaction system, or the control module is configured to control the voice interaction system to automatically play pre-stored soothing voice messages.

In one of the embodiments, the control module is further configured to record the correlation between the voice messages played by the voice interaction system and the pet's reactions; or the pet living condition detection device further includes a sound sensor and/or a camera, the control module is configured to play voice messages to sooth the pet when the sound sensor detecting abnormal sound of the pet or the camera obtain abnormal pet’s status, and the control module is configured to record the correlation between the different pet sounds obtained by the sound sensor and/or the pet's status obtained by the camera, the voice messages played by the voice interaction system, and the pet's reactions.

In one of the embodiments, the environmental adjustment device further includes an ambient light connected to the control module, the control module is further configured to automatically adjust the lighting inside the crate according to the pet's schedule; or the environmental adjustment device further includes a fragrance dispenser configured to release pet-specific fragrances; or the environmental adjustment device further includes an intelligent deodorizing device configured to deodorize the interior of the crate.

In another aspect, a pet management system is provided and includes:
the above-described pet soundproof crate for office environments and a mobile terminal, wherein the control module of the pet soundproof crate for office environments is signal-connected to the mobile terminal.

In a further aspect, a pet management system is provided and includes:
the above-described pet soundproof crate for office environments, a mobile terminal, and a server, wherein the control module of the pet soundproof crate for office environments is signal-connected to the server, and the mobile terminal is signal-connected to the server.

In the embodiment of the pet soundproof crate for office environments and the pet management system of present application, at least some of the outer walls are defined as soundproof walls to ensure excellent noise isolation for the crate, thus preventing sounds made by the pet from disrupting office order and avoiding office noise to affect mood of the pet. Additionally, pet owners can obtain the pet's living conditions via the display screen and take measures timely to maintain a comfortable environment for the pet, thereby enhancing the pet's well-being and enabling intelligent pet management.

### BRIEF DESCRIPTION OF DRAWINGS

In order to illustrate the technical solution in embodiments of the present application more clearly, the following briefly introduces accompanying drawings used in the description of the embodiments. Obviously, the accompanying drawings in the following description are only some embodiments of the present application. Those of ordinary skill in the art can obtain other accompanying drawings from these accompanying drawings without any creative efforts.

FIG. 1 is a schematic, perspective view of a pet soundproof crate for office environments according to an embodiment of the present application.

FIG. 2 is a front view of the pet soundproof crate for office environments of FIG. 1.

FIG. 3 is a back view of the pet soundproof crate for office environments of FIG. 1.

FIG. 4 is a top view of the pet soundproof crate for office environments of FIG. 1.

FIG. 5 is a cross sectional view along line A-A in FIG. 4.

FIG. 6 is a cross sectional view along lien B-B in FIG. 4.

FIG. 7 is a schematic view of the pet soundproof crate for office environments of FIG. 1 with removing a front wall.

FIG. 8 is a schematic view of the pet soundproof crate for office environments of FIG. 1 with removing outer walls.

FIG. 9 is a block diagram of part of components of the pet soundproof crate for office environments of FIG. 1.

FIG. 10 is a schematic view of a pet management system of an embodiment of the present application.

FIG. 11 is a schematic view of a pet management system of another embodiment of the present application.

### Drawing reference:

display screen: 10; top wall: 102; bottom wall: 102; side wall: 103; ventilation hole: 1031; front wall: 104; back wall: 105; sound-absorbing cotton layer: 11; soundproof plate layer: 13; double-layer soundproof structure: 15; first plate: 151; second plate: 152; cavity: 153; frame body: 17; mounting hole: 171; main frame: 172; door frame: 173; door: 18; fan: 19; cover: 21; transparent plate: 27; control module: 30; temperature sensor: 31; heating device: 32; camera: 33; mobile terminal: 34; sound sensor: 35; voice interaction system: 37; ambient light: 39; fragrance dispenser: 41; intelligent deodorizing device: 43; server: 45; pet living condition detection device: 51; environmental adjustment device: 52.

### DESCRIPTION OF EMBODIMENTS

The technical solution in the embodiments of the present application will be clearly and completely described below with reference to accompanying drawings in the embodiments of the present application. Obviously, the described embodiments are only a part of the embodiments of the present application, but not all of the embodiments. Based on the embodiments of the present application, all other embodiments obtained by those skilled in the art without any creative efforts fall within the protection scope of the present application.

Unless otherwise specified, the experimental materials and reagents used in the following embodiments are all commercially available.

For technical details or conditions not explicitly stated in the embodiments, they may be implemented according to techniques or conditions described in the literature of the field or as specified in product manuals.

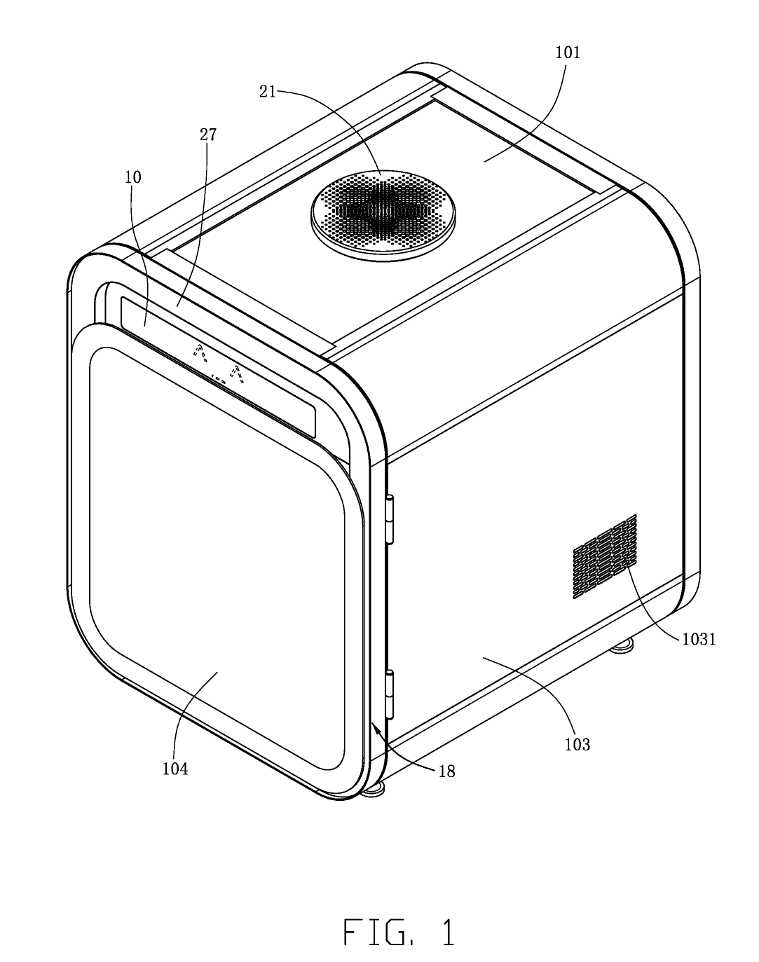

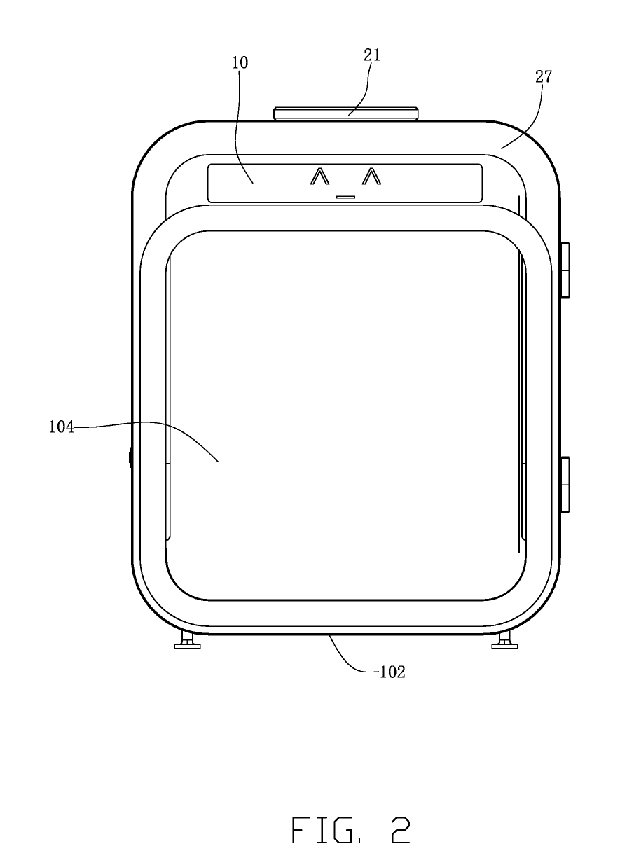

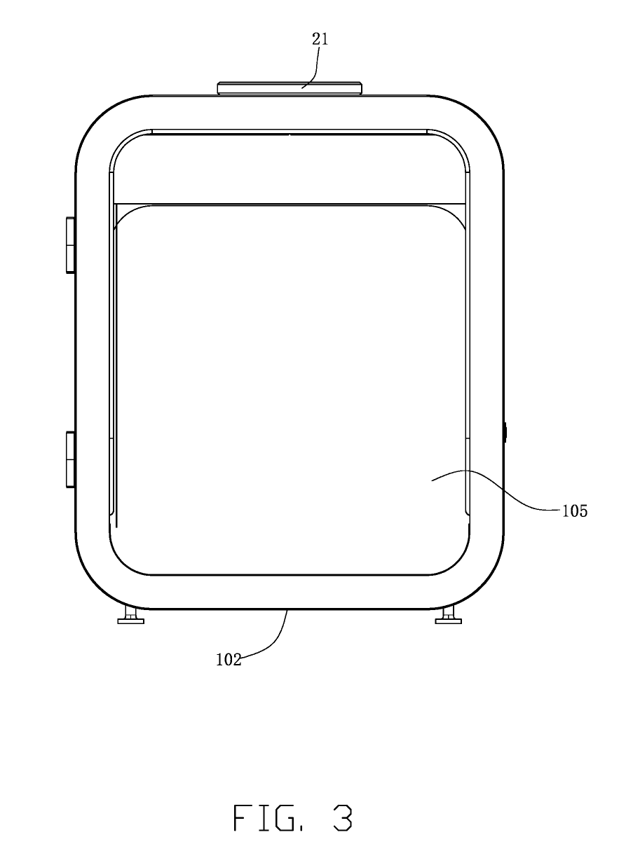

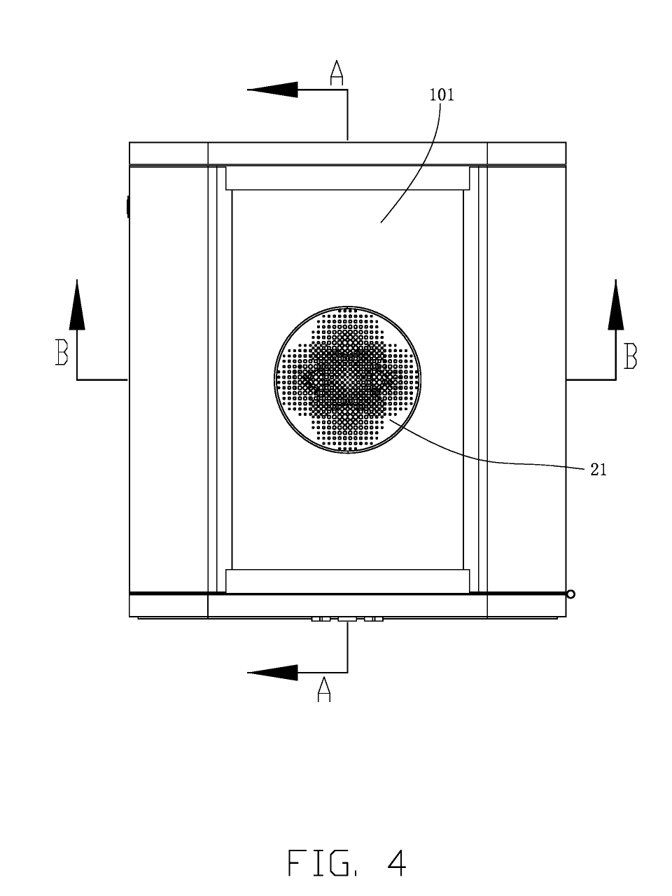

Please referring to FIGS. 1 to FIG. 4, a pet soundproof crate for office environments according to an embodiment of the present application includes a plurality of outer walls, and the plurality of outer walls enclose a receiving cavity. At least some of the plurality of outer walls defined as soundproof walls. At least one of the plurality of outer walls is equipped with a display screen 10. A pet living condition detection device 51 for monitoring pet's living conditions and an environmental adjustment device 52 are provided in the pet soundproof crate for office environments. The display screen 10 is configured to display the pet's living conditions, and the environmental adjustment device 52 is configured to adjust an internal environment of the pet soundproof crate for office environments according to the pet's living conditions.

In the embodiment of the pet soundproof crate for office environments, at least some of the outer walls are defined as soundproof walls to ensure excellent noise isolation for the crate, thus preventing sounds made by the pet from disrupting office order and avoiding office noise to affect mood of the pet. Additionally, pet owners can obtain the pet's living conditions via the display screen and take measures timely to maintain a comfortable environment for the pet, thereby enhancing the pet's well-being and enabling intelligent pet management.

In this embodiment, the pet's living conditions include environmental parameters such as temperature and humidity inside the pet soundproof crate for office environments, and a pet's status etc. The display screen 10 can show environmental parameters such as temperature and humidity inside the pet soundproof crate for office environments, and can also show the pet's status (e.g., displaying a smiley face when the pet is in a good status) etc.

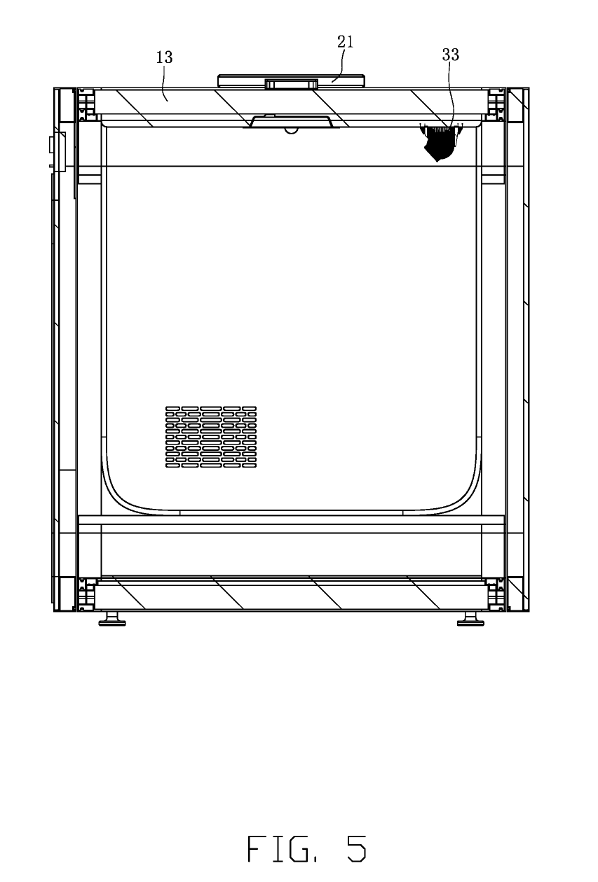

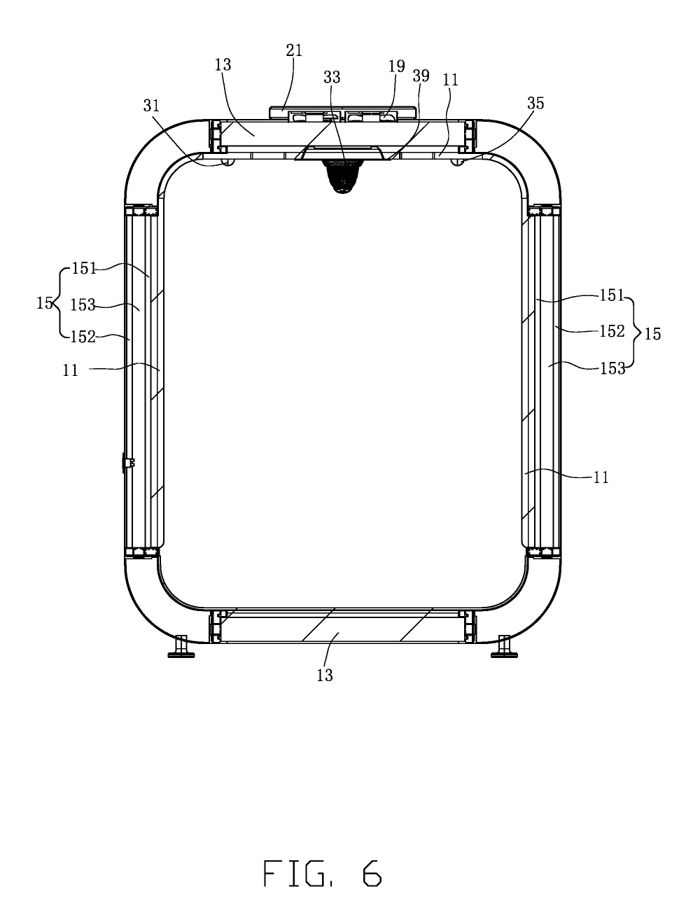

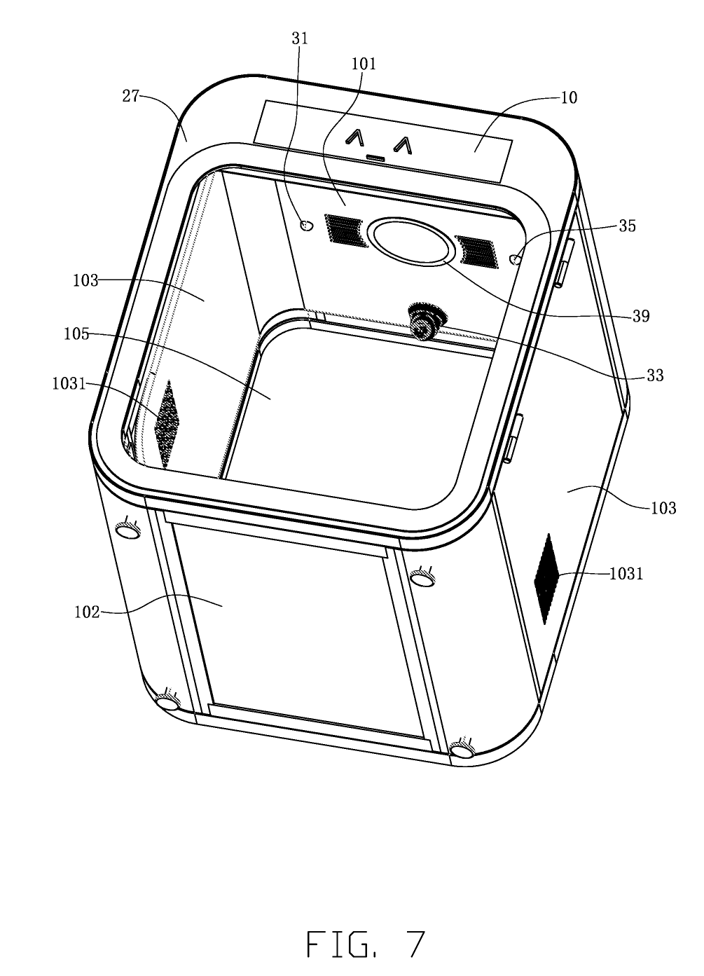

In this embodiment, with reference to FIG. 5 to FIG. 7, each of the soundproof walls is a multilayer structure and includes a sound-absorbing cotton layer 11. Specifically, the sound-absorbing cotton layer 11 is disposed on an inner side of the soundproof wall.

Specifically, the plurality of outer walls include a top wall 101, a bottom wall 102, and several side walls 103. The top wall 101 and the side walls 103 are defined as the soundproof walls. The top wall 101 further includes a soundproof plate layer 13 located at an outside of the sound-absorbing cotton layer 11. The bottom wall 102 includes a soundproof panel layer 13 to provide sound insulation. Each of the side walls 103 further includes a double-layer soundproof structure 15 located at an outside of the sound-absorbing cotton layer 11. This configuration demonstrates that both the top wall 101 and the side walls 103 employ dual-layer soundproofing structure with excellent noise isolation performance. Specifically, the double-layer soundproof structure 15 includes a first plate 151 and a second plate 152, the first plate 151 and the second plate 152 are spaced from each other, and a cavity 153 is defined between the first plate 151 and the second plate 152. The cavity 153 enhances a soundproofing effectiveness of the double-layer soundproof structure 15. It should be understood that some of the side walls 103 may alternatively be designed as transparent.

In this embodiment, the top wall 101 including the soundproof plate layer 13 and the sound-absorbing cotton layer 11 serves as a first soundproof wall, each of the side walls 103 including the double-layer soundproof structure 15 and the sound-absorbing cotton layer 11 severs as a second soundproof wall, and the bottom wall 102 including the soundproof plate layer 13 severs as a third soundproof wall.

Specifically, the outer walls further include a front wall 104 and a rear wall 105, and the front wall 104 and the rear wall 105 are made of transparent materials such as transparent glass or transparent plastic. The transparent front wall 104 and rear wall 105 enable pet owners to observe their pets inside the pet soundproof crate and facilitate to communicate with the pet, and enable the pet to have a nice visibility to enhance psychological comfort to improve a good psychological safety of the pet and reduce confinement anxiety. For improving sound insulation, the transparent material may be made of soundproof glass with superior acoustic performance. The transparent material preferably employs scratch-resistant transparent materials. It should be understood that only one of the front wall 104 and the rear wall 105 may be transparent, and the other one of the front wall 104 and the rear wall 105 may be a soundproof wall the same as the side walls 103 in an alternative embodiment. Specifically, in this embodiment, the pet soundproof crate for office environments may be cuboid-shaped. It should be understood that the pet soundproof crate for office environments may be other geometric configurations such as spherical, pyramidal, cylindrical, conical, or irregular shapes, provided that the pet soundproof crate for office environments having some outer walls being transparent and the other outer walls defined as the soundproof walls.

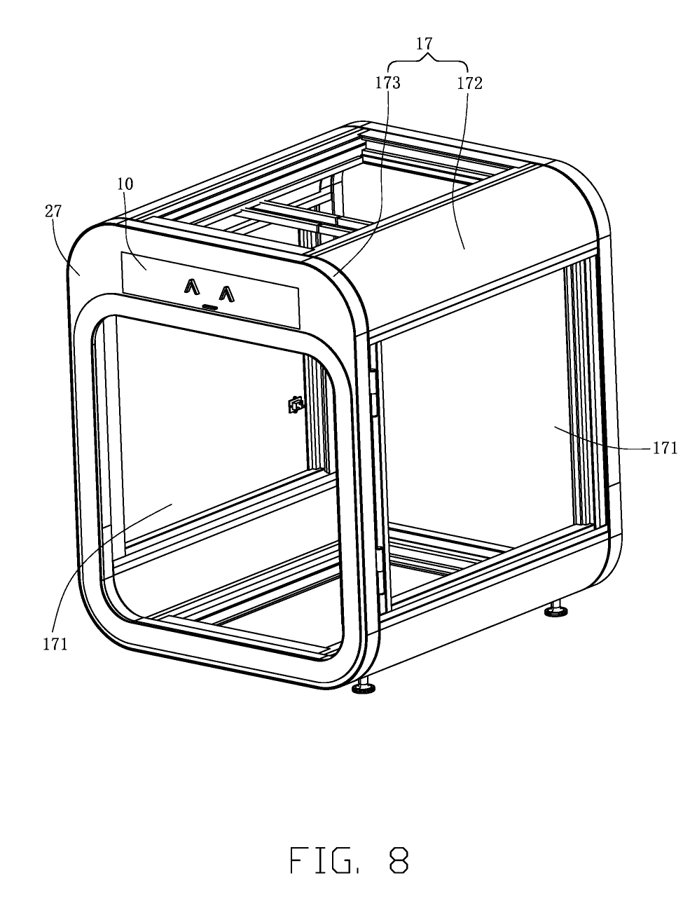

Specifically, referring to FIG. 8, the pet soundproof crate for office environments includes a frame body 17 and the frame body 17 is a frame structure to serve as a skeletal framework. Each of the top wall 101, the bottom wall 102, the side walls 103, the front wall 104, and the rear wall 105 is mounted within a corresponding mounting hole 171 defined in the frame body 17. With the modular design, the frame body 17 may be manufactured by welding or casting processes, and then the outer walls are mounted to the frame body 17, thus it is convenient to manufacture and the number of the transparent outer wall and the number of the soundproof outer wall may be determined as needed. The frame body 17 includes a main frame 172 and a door frame 173 movably connected to the main frame 172. The front wall 104 is assembled to the door frame 173 to form a door 18. Specifically, in this embodiment, the door frame 173 is pivotally connected to the main frame 172. The door 18 employs an anti-pinch safety structure to prevent injury, so as to ensure the pet to enter or exit. For example, when an object or a human body in a door gap is detected by sensors, the door may be stopped automatically or opened in reverse to avoid injuring by being clamped. Additionally, the door 18 may be opened or closed by employing either mechanical switches or intelligent access control systems.

In this embodiment, the environmental adjustment device 52 includes a fan 19, and at least one of the outer walls is provided with a fan 19. When activated, the fan 19 facilitates air circulation between the interior of the pet soundproof crate for office environments and the external environment, thereby reducing odors inside the crate. Specifically, the fan 19 may be disposed in the top wall 101. A cover 21 may be placed over the fan 19, and a plurality of ventilation holes are defined in the cover 21 to allow air to pass through. Both the sound-absorbing cotton layer 11 and the soundproof board layer 13 of the top wall 101 are made of breathable materials, which permit the passage of air. Thus, installing the fan 19 on the top wall 101 ensures air circulation without compromising the soundproofing effect. In this embodiment, the fan 19 is disposed on the outer side of the soundproof board layer 13. Specifically, the fan 19 may be a silent fan. By placing the fan 19 on the top wall 101, which is equipped with a soundproof layer, both sound insulation and air quality can be maintained.

Specifically, an air filter (not shown in FIGS) is further installed at the top wall 101 to absorb odors in the crate, preventing odors disperse to the external environments to deteriorate the air quality of the office environment during ventilation.

Specifically, ventilation holes 1031 are defined in the side walls 103 to establish convective airflow - allowing external fresh air to enter the crate through the ventilation holes 1031 and allowing odor-laden air to exhaust out from the crate to the external environments via the fan 19, thereby enhancing overall ventilation efficiency.

Specifically, the display screen 10 is disposed on the door 18. It should be understood that the display screen 10 may be disposed on the side walls 103 or the top wall 101. A transparent panel 27 may be provided to cover the display screen 10.

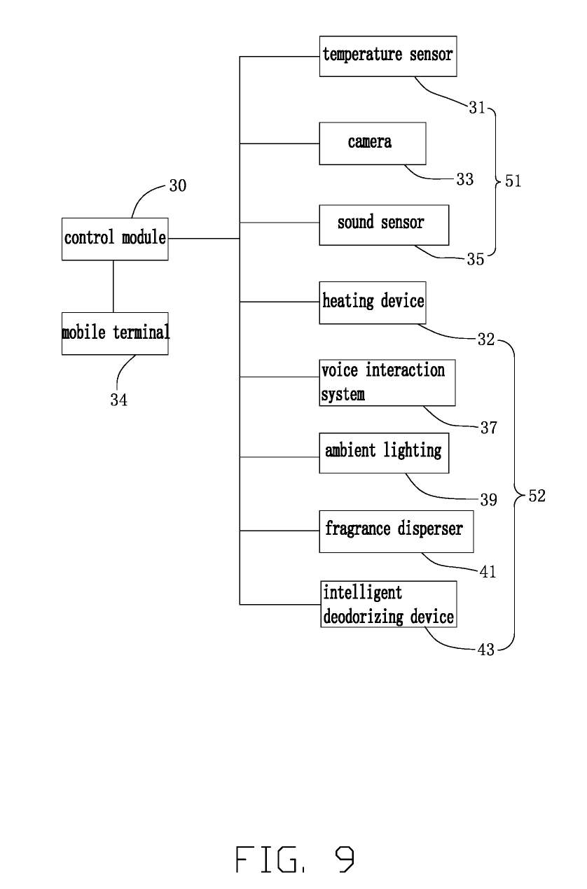

In this embodiment, referring to FIG. 9, the pet soundproof crate for office environments includes a control module 30. The control module 30 is configured to obtain the pet's living conditions detected by the pet living condition detection device 51, and to control the environmental adjustment device 52 to adjust the environment condition inside the pet soundproof crate for office environments according to the pet's living conditions. In this way, in this embodiment, the pets are allowed to be brought into office environments, enabling coexistence between humans and pets. It also enables pet owners to monitor the pets' status in real-time and provide timely feedback according to the pets' status. This not only meets the needs of coexistence between humans and pets in office environments, but also ensures that pets are in a comfortable environment.

Specifically, the pet living condition detection device 51 may include a temperature sensor 31, which is configured to detect the temperature inside the crate. The environmental adjustment device 52 may further include a heating device 32. Both the temperature sensor 31 and the heating device 32 are connected to the control module 30. When the temperature inside the crate is too high or too low, the temperature inside the crate can be adjusted by the fan 19 or the heating device 32. Specifically, when the temperature inside the crate is too high, the control module 30 can activate the fan 19 to cool down the crate. When the temperature inside the crate is too low, the control module 30 can activate the heating device to warm up the crate. Specifically, a first preset temperature and a second preset temperature are pre-stored in the control module 30, with the first preset temperature being higher than the second preset temperature. When the temperature inside the crate is greater than or equal to the first preset temperature, it indicates that the temperature inside the crate is too high. When the temperature inside the crate is less than or equal to the second preset temperature, it indicates that the temperature inside the crate is too low. By controlling the temperature inside the crate, the needs of different breeds of pets can be met, ensuring that any breed of pet can be in a comfortable environment. Depending on different requirements, the first preset temperature and the second preset temperature may be changed.

Specifically, the pet living condition detection device 51 may further include a camera 33, which is configured to obtain the pet’s status inside the crate. The control module 30 may be signal-connected to a mobile terminal 34. The control module 30 is further configured to transmit the pet's living conditions and/or the pet’s status to the mobile terminal 34. Pet owners can monitor the pet's living conditions and/or pet’s status in real-time by using the mobile terminal 34. The control module 30 can further accept control instructions from the mobile terminal 34 to control the environmental adjustment device 52, such as turning the fan 19 on or off. Specifically, the control module 30 may include a signal module, and the signal module may be signal-connected to the mobile terminal 34 through Wi-Fi, Bluetooth, cellular networks, or other means. Specifically, the camera 33 can be a 1080P HD camera, which may also support infrared night vision. Specifically, the camera 33 may be installed on the top of the pet soundproof crate for office environments, i.e., on the top wall 101. Pet owners can remotely monitor their pets' status via the mobile terminal 34, avoiding the need to frequently check on the pet. This allows pet owners to focus on their work and indirectly improves work efficiency.

Specifically, the pet living condition detection device 51 may further include a sound sensor 35, which is configured to detect whether the pet is making abnormal noises. The sound sensor 35 is connected to the control module 30 and sends a detected sound signals to the control module 30. The control module 30 is configured to generate an alert signal and trigger an alert when the pet makes abnormal noises. Specifically, when the sound sensor 35 detects that the pet is making abnormal noises (such as continuous barking), it indicates that the pet's emotions or environment may be abnormal. The control module 30 can trigger an alert to remind the pet owner to check on or comfort the pet in time with various manners. The alert signal generated by the control module 30 may be sent to the display screen 10 for display, or to an external alarm device (such as a light or sound alarm) for alerting, or to the mobile terminal 34 for reminding. Specifically, the control module 30 may store information correlating pet’s sounds with pet’s emotions. After the sound sensor 35 obtaining the pet's sound, the control module 30 may compare the obtained pet’s sound with the pre-stored information having pet’s sounds correlating with pet’s emotions to determine the pet's emotions.

Specifically, the environmental adjustment device 52 may further include a voice interaction system 37, which is connected to the control module 30. The control module 30 is also configured to receive voice messages sent from the mobile terminal 34, and the voice interaction system 37 plays the voice messages. The control module 30 may further control the voice interaction system 37 to automatically play pre-stored soothing voice messages. The voice interaction system 37 may play the pet owner's voice to communicate with the pet, or play sounds that please the pet (such as the sounds of other animals of the same species) to soothe the pet's emotions. More specifically, the control module 30 may further determine the barking level of the pet according to the sound obtained by the sound sensor 35 (factors determining the barking level may include the decibel level and pitch of the sound), and control the voice interaction system 37 to soothe the pet with different audio frequencies. The control module 30 may further record the correlation between the voice messages played by the voice interaction system 37 and the pet's reactions, thereby identifying which voice messages are more effective in improving the pet's mood. In another embodiment, the control module 30 may further record the correlation between the different pet sounds obtained by the sound sensor 35, the pet's status obtained by the camera 33, the voice messages played by the voice interaction system 37, and the pet's reactions. Therefore, an appropriate voice message can be chosen to play under a corresponding pet's emotional state.

Specifically, the environmental adjustment device 52 may further include an ambient light 39, and the ambient light 39 is connected to the control module 30. The control module 30 is further configured to automatically adjust the lighting inside the crate according to pet's circadian rhythm, ensuring that the pet is under appropriate lighting conditions. For example, when the camera 33 captures that the pet is sleeping, the control module 30 can control the ambient light 39 to dim the light. That is, the control module 30 adjusts the lighting of the ambient light 39 according to the pet's circadian rhythm to simulate the day-night cycle.

Specifically, the environmental adjustment device 52 may further include a fragrance dispenser 41, which is configured to release pet-specific fragrances to help alleviate the pet's anxiety. In one embodiment, the fragrance dispenser 41 may continuously release pet fragrances. In another embodiment, the fragrance dispenser 41 may be connected to the control module 30. When the pet's mood is detected to be poor, the control module 30 controls the fragrance dispenser 41 to release pet fragrances. Conversely, when the pet's mood is good, the control module 30 controls the device to stop releasing pet fragrances.

Specifically, the environmental adjustment device 52 further includes an intelligent deodorizing device 43, which is configured to deodorize the interior of the crate to reduce unpleasant odors. Specifically, the intelligent deodorizing device 43 may be an activated carbon filtration device or a negative ion purification device. Specifically, the intelligent deodorizing device 43 may be installed on the rear side of the pet soundproof crate for office environments, with regular replacement of the filter cartridge to ensure air quality. In one embodiment, the intelligent deodorizing device 43 may deodorize continuously. In another embodiment, the intelligent deodorizing device 43 may be connected to the control module 30. When a strong odor is detected inside the crate, the control module 30 activates the intelligent deodorizing device 43 to deodorize. When the odor inside the crate is faint or absent, the control module 30 controls the device to remain off and not perform deodorization.

In this embodiment, the pet soundproof crate for office environments may further include a urine pad (not shown in the FIGS) inside the crate, to facilitate the management of the pet's toileting needs.

In this embodiment, the pet soundproof crate for office environments may further be equipped with an automatic water dispenser (not shown in the FIGS) inside the crate, to ensure the cleanliness of the water source and prevent the pet from knocking it over.

In this embodiment, the pet soundproof crate for office environments may further be equipped with an automatic feeding system (not shown in the FIGS) inside the crate, to dispense dry food at set times.

It should be understood that the pet soundproof crate for office environments may further be equipped with pet entertainment devices (such as tunnels) inside the crate, to provide playtime for the pet.

In this embodiment, the pet soundproof crate for office environments can provide a constant and comfortable internal environment for the environmental adjustment device 52, and can also be remotely monitored and managed through the pet living condition detection device 51. It is well-suited for office environments, enhancing employee work efficiency and also improving the welfare of pets.

Certainly, the pet soundproof crate for office environments of the embodiment may also be placed at locations other than offices, such as the entrances of parks, shopping malls, or pet stores. This allows pet owners to properly accommodate their pets when entering places where pets are not allowed, such as parks or shopping malls, or to keep track of their pets' conditions when leaving them at a pet store. When the pet soundproof crate for office environments is located in public places, it may be made available to pet owners through a rental arrangement. In one rental arrangement, the pet owner may pay a rental fee to the owner of the pet soundproof crate and then open the cabin door to place the pet inside. In another rental arrangement, the pet owner may scan a code to pay the rental fee and open the door to place the pet inside.

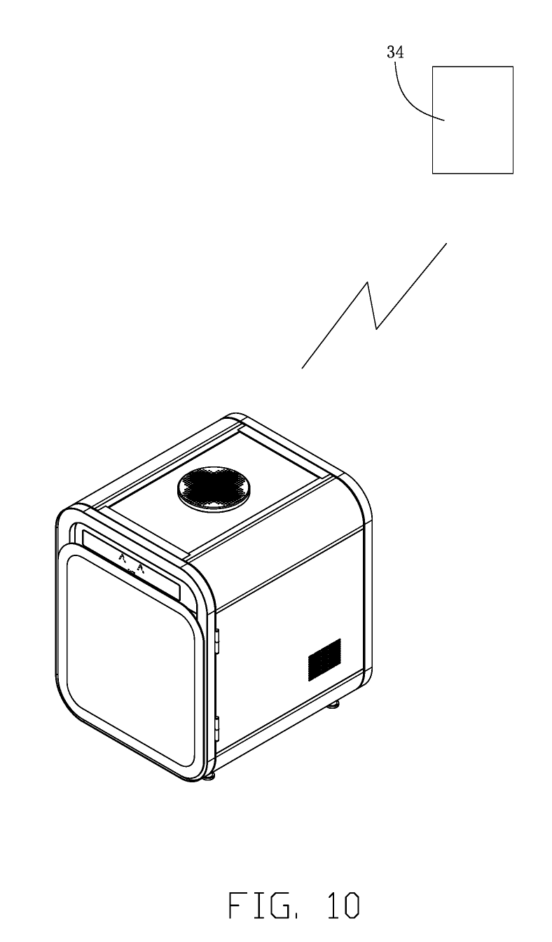

The present application may further provide a pet management system. Please referring to FIG. 10, the pet management system includes the above-mentioned pet soundproof crate for office environments and a mobile terminal 34. The control module 30 of the pet soundproof crate for office environments is signal-connected to the mobile terminal 34.

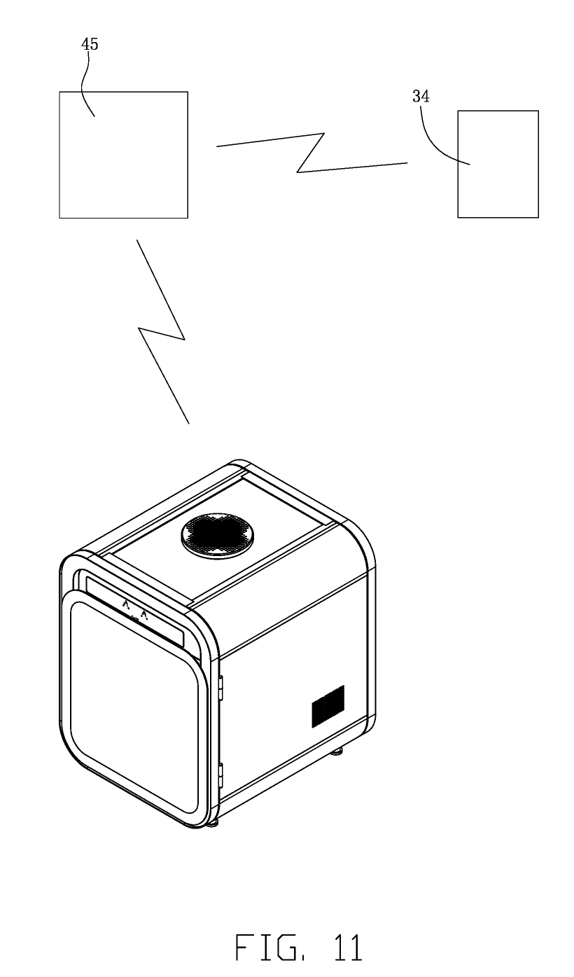

The present application may further provide another pet management system. Please referring to FIG. 11, the pet management system includes the above-mentioned pet soundproof crate for office environments, a mobile terminal 34 and a server 45. The control module 30 of the pet soundproof crate for office environments is signal-connected to the server 45 via means such as data cables, Wi-Fi, Bluetooth, or cellular networks. The mobile terminal 34 is also signal-connected to the server 45 via means such as Wi-Fi, Bluetooth, or cellular networks. The pet soundproof crate for office environments communicates information with the mobile terminal 34 via the server 45. Specifically, the correlation between the voice messages played by the voice interaction system 37 and the pet's reactions, as recorded by the control module 30 may be stored in the server 45; or the correlation among the pet’s sounds obtained by the sound sensor 35 and/or the pet's status obtained by the camera 33, the voice messages played by the voice interaction system 37, and the pet's reactions, may be stored in the server 45.

The above merely describes preferred embodiments of the present application without intention to limit the scope of the present application. Although the present application has been described in detail with reference to the foregoing embodiments, for those skilled in the art, the technical solutions described in the foregoing embodiments can still be modified, or some of the technical features can be equally replaced. Any modifications, equivalent replacements, improvements, and etc. made within the spirit and principle of the present application should be within the scope of the present application.

What is claimed is:

1. A pet soundproof crate for office environments, comprising:
a plurality of outer walls enclosing a receiving cavity, and at least some of the outer walls defined as soundproof walls;
a display screen equipped on at least one of the plurality of outer walls;
a pet living condition detection device, for detecting pet's living conditions, provided in the pet soundproof crate for office environments, and the display screen being configured to display the pet's living conditions; and
an environmental adjustment device, for adjusting the internal environment of the pet soundproof crate for office environments according to the pet’s living conditions, provided in the pet soundproof crate for office environments.

2. The pet soundproof crate for office environments of claim 1, wherein each of the soundproof walls is a multilayer structure, and each of the soundproof walls comprises a sound-absorbing cotton layer.

3. The pet soundproof crate for office environments of claim 2, wherein the plurality of outer walls comprise a top wall, a bottom wall, and side walls, the side walls are defined as the soundproof walls, the sound-absorbing cotton layer is located at an inner side of the soundproof wall, each of the side walls comprises a double-layer soundproof structure located at an outside of the sound-absorbing cotton layer, the double-layer soundproof structure comprises a first plate and a second plate, the first plate and the second plate are spaced from each other so as to define a cavity between the first plate and the second plate.

4. The pet soundproof crate for office environments of claim 3, the top wall is defined as the soundproof wall, the top wall further comprises a soundproof panel layer, the soundproof plate layer is located at an outside of the sound-absorbing cotton layer; the bottom wall only comprises the soundproof panel layer.

5. The pet soundproof crate for office environments of claim 4, the environmental adjustment device comprises a fan disposed in the top wall, a cover is placed over the fan, a plurality of ventilation holes are defined in the cover to allow air to pass through, and both the sound-absorbing cotton layer and the soundproof board layer of the top wall are made of breathable materials.

6. The pet soundproof crate for office environments of claim 3, wherein the outer walls further comprise a front wall and a rear wall, and at least one of the front wall and the rear wall is made of transparent material.

7. The pet soundproof crate for office environments of claim 1, wherein the pet soundproof crate for office environments comprises a frame body and the frame body is a frame structure to serve as a skeletal framework, each of the plurality of outer walls are all mounted within a corresponding mounting hole defined in the frame body; the frame body comprises a main frame and a door frame movably connected to the main frame, and one of the plurality of outer walls is assembled on the door frame to form a door.

8. The pet soundproof crate for office environments of claim 1, wherein the pet soundproof crate for office environments further comprises a control module configured to obtain the pet's living conditions detected by the pet living condition detection device and to control the environmental adjustment device to adjust the environment inside the pet soundproof crate for office environments according to the pet's living conditions.

9. The pet soundproof crate for office environments of claim 8, wherein the pet living condition detection device comprises a temperature sensor configured to detect the temperature inside the crate, the environmental adjustment device comprises a fan and a heating device, the temperature sensor and the heating device are connected to the control module, and the control module is configured to adjust the temperature inside the crate can be adjusted by the fan or the heating device when the temperature inside the crate is too high or too low.

10. The pet soundproof crate for office environments of claim 8, wherein the pet living condition detection device further comprises a camera for obtaining a pet's status inside the crate, and the control module is signal-connected to a mobile terminal and configured to send the pet's living conditions and/or the pet’s status to the mobile terminal.

11. The pet soundproof crate for office environments of claim 8, wherein the pet living condition detection device further comprises a sound sensor configured to detect whether the pet is making abnormal noises, the sound sensor is connected to the control module and configured to send a detected sound signals to the control module, and the control module is configured to generate an alert signal and trigger an alert when the pet makes abnormal noises.

12. The pet soundproof crate for office environments of claim 11, wherein the control module stores information correlating pet’s sounds with pet’s emotions, and the control module is further configured to compare an obtained pet’s sound with the pre-stored information having pet’s sounds correlating with pet’s emotions to determine the pet's emotions.

13. The pet soundproof crate for office environments of claim 11, wherein the alert signal generated by the control module is sent to the display screen to be displayed on the display screen; or the alert signal generated by the control module is sent to an external alarm device for alerting; or the alert signal generated by the module is sent to a mobile terminal for a reminder.

14. The pet soundproof crate for office environments of claim 8, wherein the environmental adjustment device further comprises a voice interaction system connected to the control module;
wherein the control module is configured to receive voice messages transmitted from a mobile terminal and the voice interaction system plays the voice messages; and/or the control module is configured to control the voice interaction system to automatically play pre-stored soothing voice messages.

15. The pet soundproof crate for office environments of claim 14, wherein the control module is further configured to record the correlation between the voice messages played by the voice interaction system and the pet's reactions; or the pet living condition detection device further comprises a sound sensor to detect obtain pet’s sound and/or a camera to obtain the pet’s status, the control module is configured to play voice messages to soothe the pet when the sound sensor detects abnormal pet’s sound or the camera obtains abnormal pet’s status, and the control module is configured to record the correlation between the pet’s sounds obtained by the sound sensor and/or the pet's status obtained by the camera, the voice messages played by the voice interaction system, and the pet's reactions.

16. The pet soundproof crate for office environments of claim 8, wherein the environmental adjustment device further comprises an ambient light connected to the control module, the control module is further configured to automatically adjust the lighting inside the crate according to the pet's schedule; or the environmental adjustment device further comprises a fragrance dispenser configured to release pet-specific fragrances; or the environmental adjustment device further comprises an intelligent deodorizing device configured to deodorize the interior of the crate.

17. A pet management system, comprising:
the pet soundproof crate for office environments according to claim 8 and a mobile terminal, wherein the control module of the pet soundproof crate for office environments is signal-connected to the mobile terminal.

18. A pet management system, comprising:
the pet soundproof crate for office environments according to claim 8, a mobile terminal, and a server, wherein the control module of the pet soundproof crate for office environments is signal-connected to the server, and the mobile terminal is signal-connected to the server.
